SEQUENCE LISTING (7 sequences)

SEQ ID NO: 1 - protein, 8 aa, synthetic construct
GYTFTEYT

SEQ ID NO: 2 - protein, 8 aa, synthetic construct
INPNNGGT

SEQ ID NO: 3 - protein, 11 aa, synthetic construct
ARRGLRPLFDY

SEQ ID NO: 4 - protein, 6 aa, synthetic construct
QNVGTN

SEQ ID NO: 5 - protein, 8 aa, synthetic construct
QQYNSYLT

SEQ ID NO: 6 - protein, 118 aa, synthetic construct
EVQLQQSGPELVKPGASVKISCKTSGYTFTEYTIHWVKQSHGKSLEYIGGINPNNGGTSYKQKFEGKATLTVDKSSSTAYMELRSLTSEDSAVYYCARRGLRPLFDYWGQGTTLTVSS

SEQ ID NO: 7 - protein, 107 aa, synthetic construct
DIVMTQSQKFMSTSVGDRVSVTCKASQNVGTNVAWYQQKPGQSPKVLIYSASSRYSGVPDRFTGSGSGTDFTLTISNVQSEDLAEYFCQQYNSYLTFGAGTKLELKR

(19) United States
(12) Patent Application Publication
(10) Pub. No.: US 2024/0369556 A1
(43) Pub. Date: Nov. 7, 2024

(54) ISOLATED MONOCLONAL ANTIBODY, ISOLATED ANTIGEN BINDING FRAGMENT THEREOF, COMPOSITION, METHOD OF ELICITING IMMUNE RESPONSE AGAINST ANTIGENIC EPITOPE OF FLAVIVIRUS IN SUBJECT, METHOD FOR DETECTING FLAVIVIRUS, POLYPEPTIDE, AND ANTIBODY-CELL CONJUGATION

(71) Applicant: CHANCE BIOTECHNOLOGY, INC., Taipei city (TW)

(72) Inventors: Li-Kuang CHEN, Hualien City (TW); Chuan CHIN, Taoyuan City (TW); Hui-Hua YANG, Hualien City (TW)

(21) Appl. No.: 18/312,737

(22) Filed: May 5, 2023

(51) Int. Cl. G01N 33/569; C07K 16/10

(52) U.S. Cl. CPC G01N 33/56983; C07K 16/1081; C07K 2317/33; C07K 2317/565; C07K 2317/76; G01N 2333/185; G01N 2469/10

(57) ABSTRACT

An isolated monoclonal antibody includes a heavy chain variable domain (V_{H}) and a light chain variable domain (V_{L}). The V_{H} includes a heavy chain complementary determining region (HCDR)1 of SEQ ID NO: 1, a HCDR2 of SEQ ID NO: 2, and HCDR3 of SEQ ID NO: 3. The V_{L} includes a light chain complementary determining region (LCDR)1 of SEQ ID NO: 4, a LCDR2 having an amino acid sequence of SAR, and a LCDR3 of SEQ ID NO: 5. The isolated monoclonal antibody specifically binds to a glycosylated envelope (E) protein of a flavivirus and neutralizes the flavivirus.

### SEQUENCE LISTING XML

A sequence listing XML submitted as an xml file via EFS-WEB is incorporated herein by reference. The sequence listing XML file submitted via EFS-WEB with the name “CP-5888-US_SEQ_LIST.xml” was created on May 1, 2023, which is 7,309 bytes in size.

### BACKGROUND

### Technical Field

The present disclosure relates to an isolated monoclonal antibody and use thereof. More particularly, the present disclosure relates to an isolated monoclonal antibody for neutralizing a flavivirus and use thereof.

### Description of Related Art

The genus Flavivirus of the family Flaviviridae includes over 70 viruses, which is enveloped and positive-sense single stranded RNA virus with a genome of approximately 9.4-13 kb in length, and the virion diameter thereof is about 50 nm. Of these viruses, 34 are mosquito borne, 17 are tick borne, and 22 are zoonotic agents transmitted with now known vector. Forty species of the flavivirus family have been associated with human disease. Yellow fever, dengue fever, and Japanese encephalitis are the most important arboviral infectious diseases across every continent and have been legitimized into Class I and Class III transmissible disease in current issue of Regulation of Prevention and Control for Transmission Disease at Taiwan.

So far, there is no specific drug for the treatment of diseases caused by flaviviruses. In terms of infection control, some flaviviruses such as yellow fever virus and JEV have vaccines that can be administered. However, epidemics caused by other flaviviruses, such as dengue fever, can only be stopped by killing the vector insects as soon as possible after early and accurate diagnosis.

In addition, to determine whether there is virus antigen in the specimen or to determine whether there is anti-virus antibody in the patient's serum during diagnosis, it is necessary to have the necessary reagent to identify the virus antigen-virus antigen-specific antibody. To reduce misdiagnosis, it is necessary to use purified monoclonal antibodies specific to viral antigens with extremely low background values. Unfortunately, there are no finished products of this kind on the market, and the few monoclonal antibodies that can be obtained from ATCC have poor diagnostic results due to their poor binding ability.

### SUMMARY

According to one aspect of the present disclosure, an isolated monoclonal antibody includes a heavy chain variable domain (V_{H}) and a light chain variable domain (V_{L}). The V_{H} includes a heavy chain complementary determining region (HCDR)1 of SEQ ID NO: 1, a HCDR2 of SEQ ID NO: 2, and HCDR3 of SEQ ID NO: 3. The V_{L} includes a light chain complementary determining region (LCDR)1 of SEQ ID NO: 4, a LCDR2 having an amino acid sequence of SAS, and a LCDR3 of SEQ ID NO: 5. The isolated monoclonal antibody specifically binds to a glycosylated envelope (E) protein of a flavivirus and neutralizes the flavivirus.

According to another aspect of the present disclosure, an isolated antigen binding fragment of the isolated monoclonal antibody according to the aforementioned aspect.

According to still another aspect of the present disclosure, a method of eliciting an immune response against an antigenic epitope of a flavivirus in a subject includes administering a therapeutically effective amount of the isolated monoclonal antibody according to the aforementioned aspect to the subject, thereby eliciting the immune response.

According to yet another aspect of the present disclosure, a composition includes the isolated monoclonal antibody according to the aforementioned aspect and a pharmaceutically acceptable carrier.

According to still another aspect of the present disclosure, a method of eliciting an immune response against an antigenic epitope of a flavivirus in a subject includes administering an immunogenically effective amount of the composition according to the aforementioned aspect to the subject, thereby eliciting the immune response against dengue virus serotype 1, dengue virus serotype 2, dengue virus serotype 3, dengue virus serotype 4, Japanese encephalitis virus (JEV), Kunjin virus, yellow fever virus, West Nile virus or Zika virus.

According to yet another aspect of the present disclosure, a method for detecting a flavivirus includes following steps. A sample from a subject is provided. The sample is contacted with the isolated monoclonal antibody according to the aforementioned aspect and then a binding reaction is performed. Then, whether the sample has an antibody-virus complex is detected.

According to still another aspect of the present disclosure, a polypeptide includes the isolated monoclonal antibody according to the aforementioned aspect.

According to yet another aspect of the present disclosure, an antibody-cell conjugation includes a cell and the polypeptide according to the aforementioned aspect conjugated to a surface of the cell. The cell is an immune cell or a stromal cell. The polypeptide specifically binds to a glycosylated envelope (E) protein of a flavivirus.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1 shows Western Blotting analysis of an isolated monoclonal antibody of present disclosure.

FIG. 2A shows analysis result of immunofluorescence staining by the isolated monoclonal antibody of present disclosure on cells infected with dengue virus serotype 1.

FIG. 2B shows analysis result of immunofluorescence staining by the isolated monoclonal antibody of present disclosure on cells infected with dengue virus serotype 2.

FIG. 2C shows analysis result of immunofluorescence staining by the isolated monoclonal antibody of present disclosure on cells infected with dengue virus serotype 3.

FIG. 2D shows analysis result of immunofluorescence staining by the isolated monoclonal antibody of present disclosure on cells infected with dengue virus serotype 4.

FIG. 2E shows analysis result of immunofluorescence staining by the isolated monoclonal antibody of present disclosure on cells infected with Japanese encephalitis virus.

FIG. 2F shows analysis result of immunofluorescence staining by the isolated monoclonal antibody of present disclosure on cells infected with Kunjin virus.

FIG. 2G shows analysis result of immunofluorescence staining by the isolated monoclonal antibody of present disclosure on cells infected with West Nile virus.

FIG. 2H shows analysis result of immunofluorescence staining by the isolated monoclonal antibody of present disclosure on cells infected with Zika virus.

FIG. 2I shows analysis result of immunofluorescence staining by the isolated monoclonal antibody of present disclosure on cells infected with yellow fever virus.

FIG. 2J shows analysis result of immunofluorescence staining by the isolated monoclonal antibody of present disclosure on mock cells.

### DETAILED DESCRIPTION

An isolated monoclonal antibody, an isolated antigen binding fragment thereof, a composition including the isolated monoclonal antibody, a polypeptide including the isolated monoclonal antibody, an antibody-cell conjugation including the polypeptide, a method of eliciting an immune response against an antigenic epitope of a flavivirus in a subject, and a method for detecting the flavivirus are provided.

The term “antibody” can be a full antibody with Fc or antigen binding portion thereof. The term “antibody” also includes all classes of antibodies, including IgG, IgA, IgM, IdD and IgE. The term “antibody” also includes variants, fusions and derivatives of any defined antibodies and antigen binding portions thereof. An antibody according to the present disclosure is obtainable by a method including a step of immunizing a mammal, such as a human, a monkey, a rabbit or a mouse; and/or by an in vitro method, for example including a phage display selection step, as will be well known to those skilled in the art.

The term “antigen binding fragment” can be a Fv fragment; a Fab-like fragment (e.g. a Fab fragment, a Fab′ fragment, a F(ab′)2 fragment, Fv or scFv fragments); or a domain antibody. The antigen binding fragment can be derived from the linear amino acid sequence present in an intact antibody, or can include a set of non-consecutive amino acids, optionally interspersed with other amino acids, for example can include particular amino acids that are required for contact with an epitope, but can for example not comprise the amino acids required for the framework of a native antibody, which, in some cases, can be replaced by a heterologous scaffold protein, for example.

By neutralize which mean reduce the ability of the virus to infect previously uninfected cells. The person skilled in the art will be well aware of suitable techniques to monitor viral neutralizing ability.

The term “effector molecule” can be a portion of a chimeric molecule that is intended to have a desired effect on a cell or protein to which the chimeric molecule is targeted. Effector molecule is also known as an effector moiety, therapeutic agent or diagnostic agent, or similar terms.

Reference will now be made in detail to the present embodiments of the present disclosure, examples of which are illustrated in the accompanying drawings.

### Examples

The flavivirus genome contains only one open reading frame (ORF) flanked by 5′ and 3′ untranslated regions (UTRs), and some flaviviruses, such as Japanese encephalitis virus (JEV) and West Nile virus (WNV) have −1 open reading frame shift events during translation. The ORF encodes a polyprotein that is processed into three structural proteins, a nucleocapsid protein (C), a precursor membrane glycoprotein (prM) and a glycosylated envelope protein (E), as well as seven non-structural (NS) proteins, NS1, NS2A/B, NS3, NS4A, 2K, NS4B, and NS5, by viral (NS2B-NS3) or host proteases (host signal peptidase and host furin). The C protein is responsible for encapsidation to protect the genetic material. PrM protein, which is formed by protease hydrolysation during late viral infection, participates in forming the viral envelope and plays an important role in maintaining the E protein's spatial structure. Both prM and E protein form the surface structure of virions. The surface structural E protein facilitates membrane fusion between the virus and host cell, and is the primary viral protein against which neutralizing antibodies are produced and is indispensable in flavivirus biology.

The isolated monoclonal antibody of the present disclosure includes a heavy chain variable domain (V_{H}) and a light chain variable domain (V_{L}). The V_{H} includes a heavy chain complementary determining region (HCDR)1 of SEQ ID NO: 1, a HCDR2 of SEQ ID NO: 2, and HCDR3 of SEQ ID NO: 3. The V_{L} includes a light chain complementary determining region (LCDR)1 of SEQ ID NO: 4, a LCDR2 having an amino acid sequence of SAS (SerAlaSer), and a LCDR3 of SEQ ID NO: 5. The isolated monoclonal antibody specifically binds to a glycosylated envelope (E) protein of the flavivirus and neutralizes the flavivirus. The V_{H} can have an amino acid sequence of SEQ ID NO: 6 and the V_{L} can have an amino acid sequence of SEQ ID NO: 7. The isolated monoclonal antibody can be an IgG, an IgM or an IgA. The isolated monoclonal antibody can be linked to an effector moiety, wherein the effector moiety is a toxin or a detectable label. Preferably, the detectable label is a fluorescent label, an enzymatic label or a radioactive label. The isolated antigen binding fragment of the isolated monoclonal antibody of present disclosure can be a Fab fragment, a Fab′ fragment, a F(ab′)2 fragment, a single chain Fv protein (scFv) fragment or a disulfide stabilized Fv protein (dsFv).

The method of eliciting the immune response against the antigenic epitope of the flavivirus in the subject includes administering a therapeutically effective amount of the isolated monoclonal antibody of the present disclosure to the subject, thereby eliciting the immune response. The flavivirus can be dengue virus serotype 1, dengue virus serotype 2, dengue virus serotype 3, dengue virus serotype 4, Japanese encephalitis virus, Kunjin virus, yellow fever virus, West Nile virus or Zika virus. The subject can be a mammal.

The composition of the present disclosure includes the isolated monoclonal antibody of the present disclosure and a pharmaceutically acceptable carrier. The method of eliciting the immune response against the antigenic epitope of the flavivirus in the subject includes administering an immunogenically effective amount of the composition of the present disclosure to the subject, thereby eliciting the immune response against dengue virus serotype 1, dengue virus serotype 2, dengue virus serotype 3, dengue virus serotype 4, Japanese encephalitis virus (JEV), Kunjin virus, yellow fever virus, West Nile virus or Zika virus. The subject can be a mammal.

The method for detecting the flavivirus includes following steps. A sample from a subject is provided. The sample is contacted with the isolated monoclonal antibody according to the aforementioned aspect and then a binding reaction is performed. Then, whether the sample has an antibody-virus complex is detected.

The polypeptide of the present disclosure the isolated monoclonal antibody of the present disclosure. The antibody-cell conjugation of the present disclosure includes a cell and the polypeptide according to the aforementioned aspect conjugated to a surface of the cell. The cell is an immune cell or a stromal cell. The polypeptide specifically binds to a glycosylated envelope (E) protein of the flavivirus. The immune cell can be a T cell or a natural killer (NK) cell.

### Materials and Methods

### I. Generation and Characterization of the Isolated Monoclonal Antibody of Present Disclosure

Six-to eight-week-old BALB/c mice were immunized intraperitoneally with West Nile virus mixed with an equal volume of Freund's complete adjuvant for first inoculation, and incomplete Freund's adjuvant was used for subsequent boosting. West Nile virus-specific antisera were obtained from the mice after three consecutive challenges. For generation of monoclonal antibodies, splenocytes were fused with Sp2-0 myeloma cells and selected. The hybridoma secreting specific antibodies were identified by immunofluorescence assay using West Nile virus-infected Vero cell lysates as antigen source. Single-cell clones were generated by limiting dilution and the cells were injected into incomplete Freund's adjuvant-primed BALB/c mice for ascitic fluid production.

### II. Virus Infection

To infect with dengue virus serotype 1, dengue virus serotype 2, dengue virus serotype 3 and dengue virus serotype 4, monolayers of the BHK-21 cells grown in 25-cm tissue culture flask were initially adsorbed with dengue virus serotype 1 to dengue virus serotype 4 at a CCID₅₀ (cell culture infective dose 50%) of 500 for 1 hour at 37° C., respectively. After adsorption, the unbound viruses were removed by three gentle washings with serum-free RPMI 1640 medium. Fresh medium containing 2% FCS was added for further incubation at 37° C.

To infect with Kunjin virus, West Nile virus, Zika virus and yellow fever virus, monolayers of the BHK-21 and Vero cells grown in 25-cm tissue culture flask were initially adsorbed with the flaviviruses at a CCID₅₀ of 100 for 1 hour at 37° C., respectively. After adsorption, the unbound viruses were removed by three gentle washings with serum-free RPMI 1640 medium. Fresh medium containing 2% FCS was added for further incubation at 37° C.

Please refer to Table 1, which shows flavivirus strains used in the experiment.

TABLE 1

| Flavivirus | Strain |
|---|---|
| Dengue virus serotype 1 | HAWAIIAN |
| Dengue virus serotype 2 | PL046 |
| Dengue virus serotype 3 | H-87 |
| Dengue virus serotype 4 | H-241 |
| Japanese encephalitis virus | NT109 |
| Kunjin virus | UVE |
| Yellow fever virus | 17D |
| West Nile virus | VR-1510 |
| Zika virus | PRVABC59 |

### III. Western Immunoblot Analysis

Cell lysates of cells infected with West Nile virus were mixed with an equal volume of the sample buffer (50 mM Tris-HCl, pH 6.8, 100 mM dithiothreitol (DTT), 2% SDS, 0.1% bromophenol blue, 10% glycerol) lacking 2-mercaptoethanol with or without boiling, separated by SDS-PAGE, and transferred to nitrocellulose membrane (Hybond-C Super; Amersham). The non-specific antibody-binding sites were blocked with 5% skimmed milk in PBS, and the filters were incubated with the isolated monoclonal antibody of the present at room temperature for 1 hour. The dilution of antibody used was 1:1,000. After washing with phosphate-buffered saline (PBS), the blot was then treated with horseradish peroxidase-conjugated goat anti-mouse immunoglobulin (Merck Millipore) and developed with 4-chloro-1-naphthol.

### IV. Indirect Immunofluorescence Assay

Cells infected with different flaviviruses were fixed by acetone/methanol (vol/vol 1:1) solution for 10 minutes, stained with the isolated monoclonal antibody of the present disclosure at 37° C., 5% CO₂ incubator for 1 hour. The dilution of antibody used was 1:1,000. After washing with phosphate-buffered saline (PBS), the cells were reacted with goat anti-mouse IgG fluorescein-conjugated secondary antibody (Merck Millipore) and examined under an Olympus fluorescent microscope.

### V. Qualitative Cross-Reactivity of the Isolated Monoclonal Antibody of Present Disclosure to Different Flavivirus Antigens

Whether the isolated monoclonal antibody of the present disclosure cross-react with other flavivirus antigens was determined by immunoblotting. Cells infected with different flaviviruses were dissolved with lysis buffer, then proteins thereof were separated by SDS-PAGE and transferred to Nitrocellulose membrane (Hybond-C Super, Amersham), and non-specific antibody-binding sites were blocked by 5% skimmed milk powder in PBS. After adding the isolated monoclonal antibody of the present disclosure and HP enzyme-conjugated goat anti-mouse immunoglobulin for reaction, the conversion substrate 4-chloro-1-naphthol is colored.

### Results

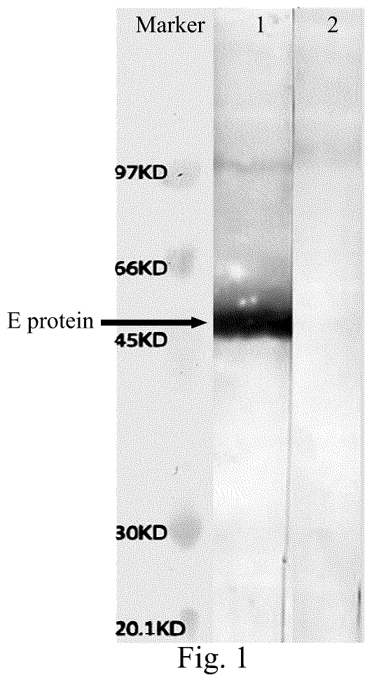

Reference is made to FIG. 1, which shows Western Blotting analysis of the isolated monoclonal antibody of present disclosure, wherein “1” represents cells infected with West Nile virus, and “2” represents mock cells. In FIG. 1, the isolated monoclonal antibody of present disclosure acts on the E protein with a molecular weight of 50 kDa. The result indicates that the isolated monoclonal antibody of present disclosure specifically binds to E protein of the flavivirus.

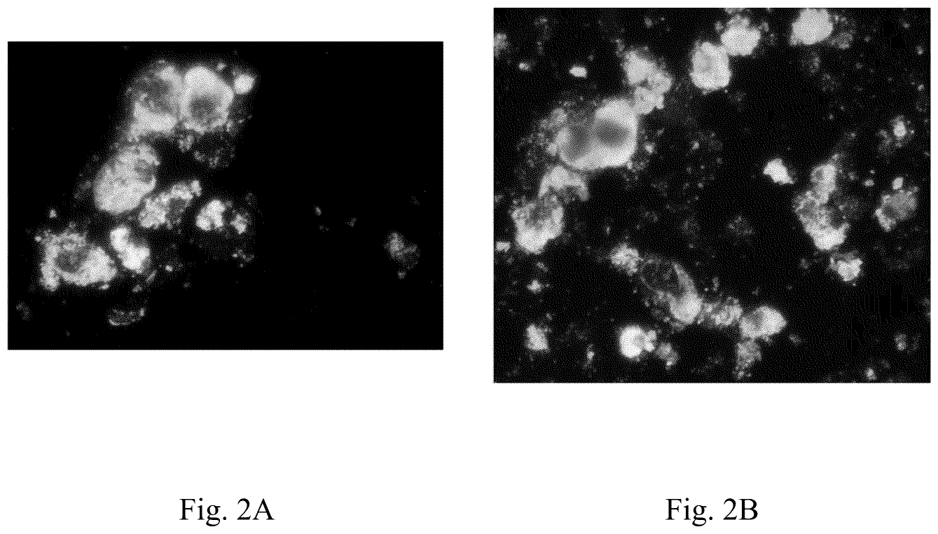

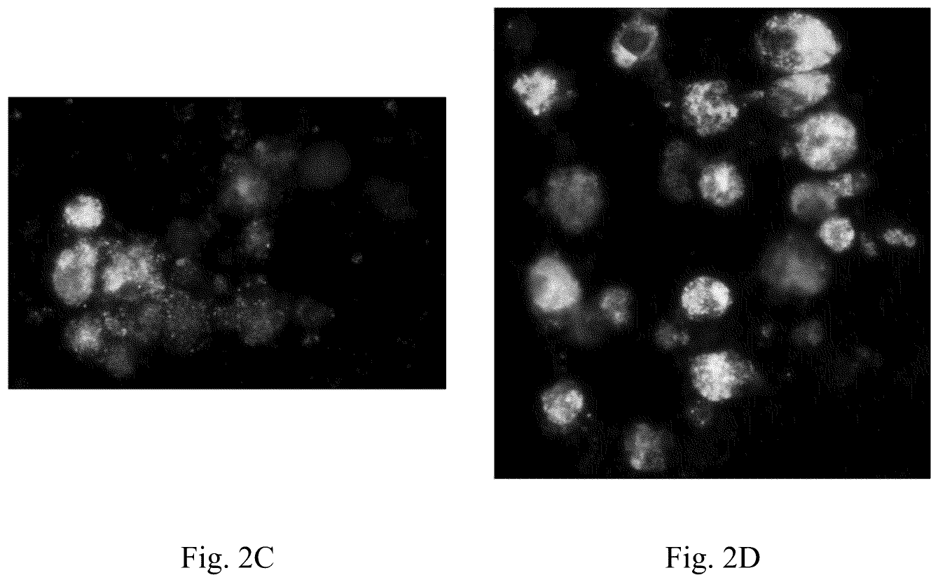

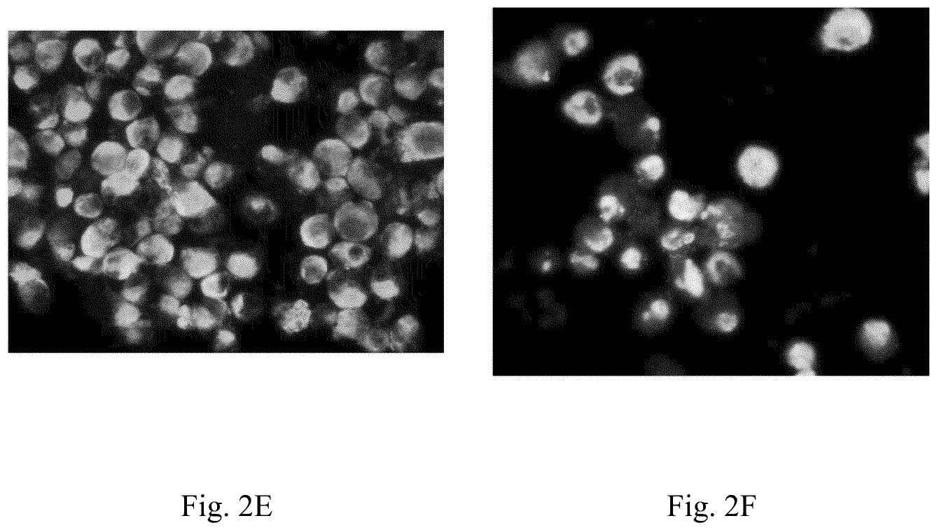

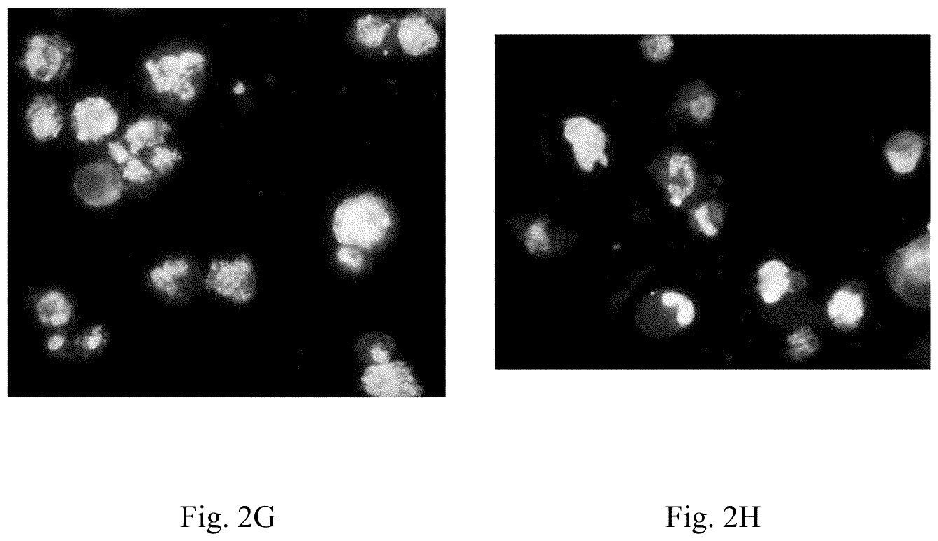

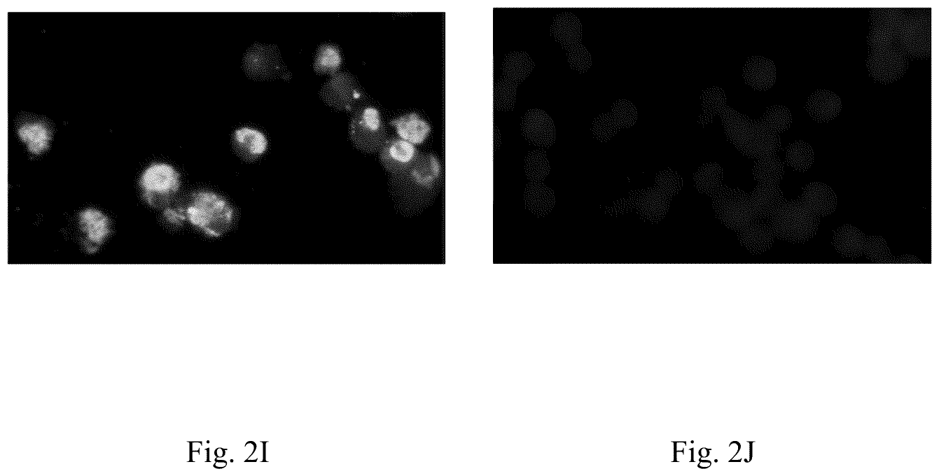

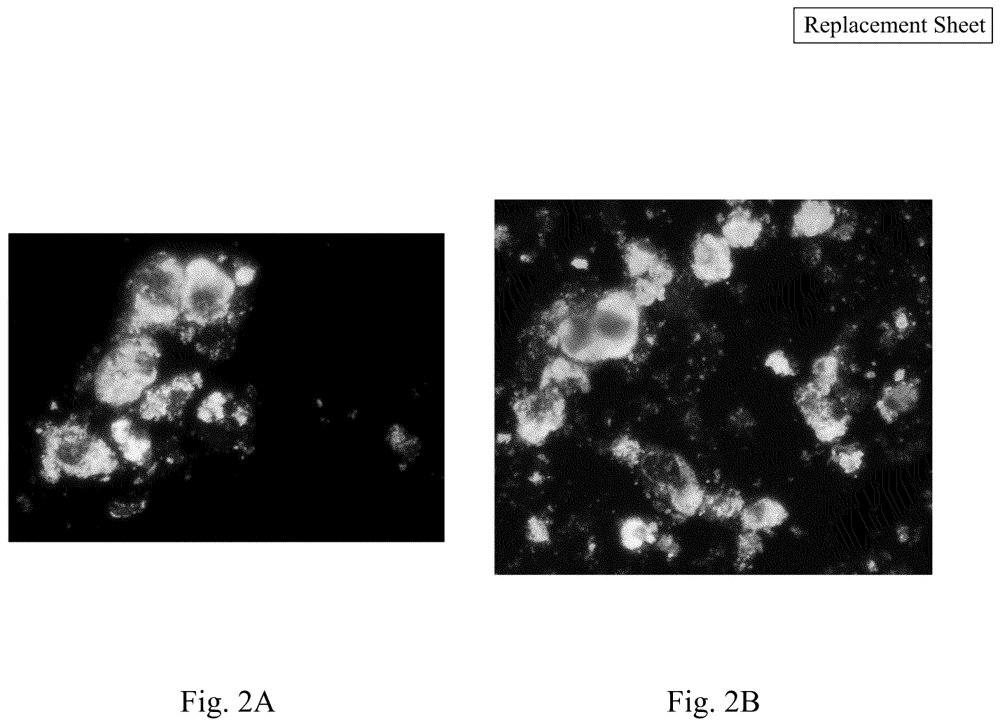

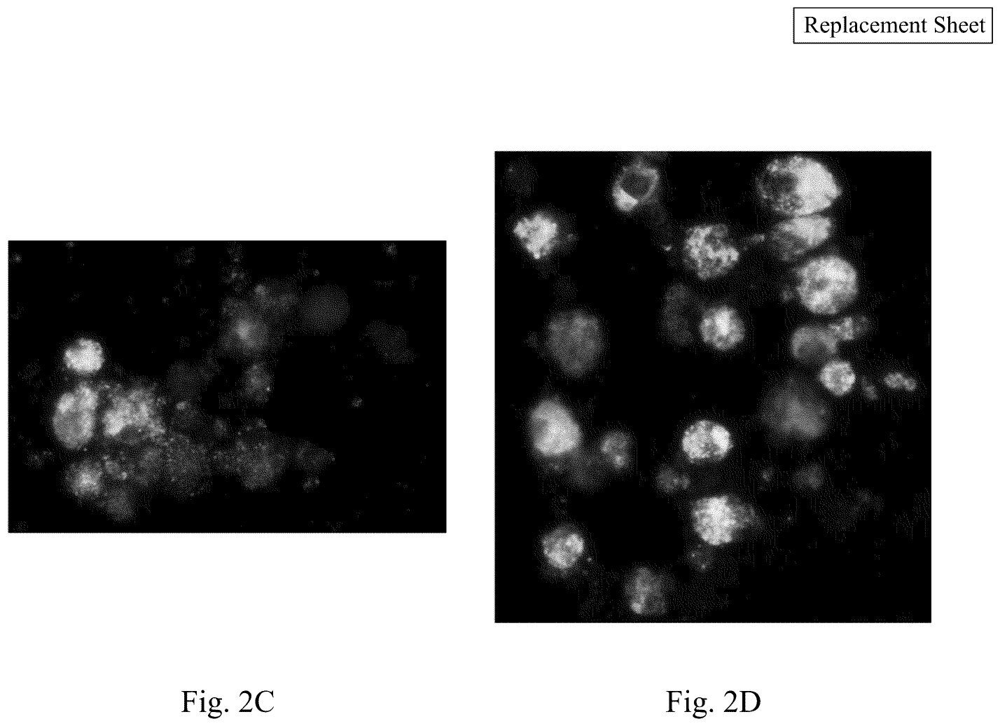

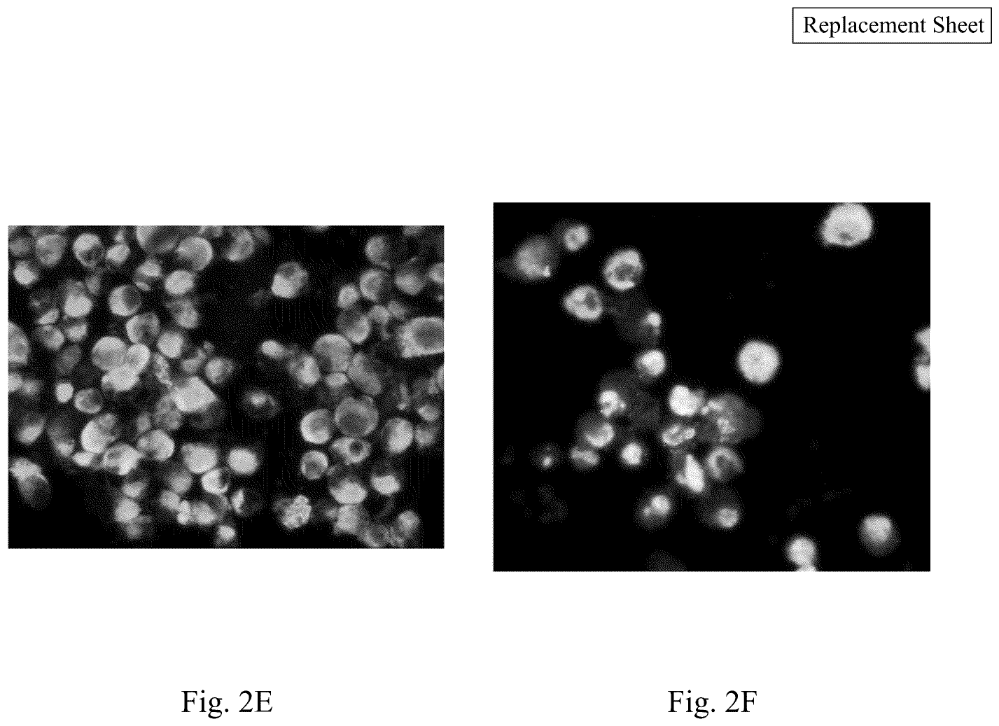

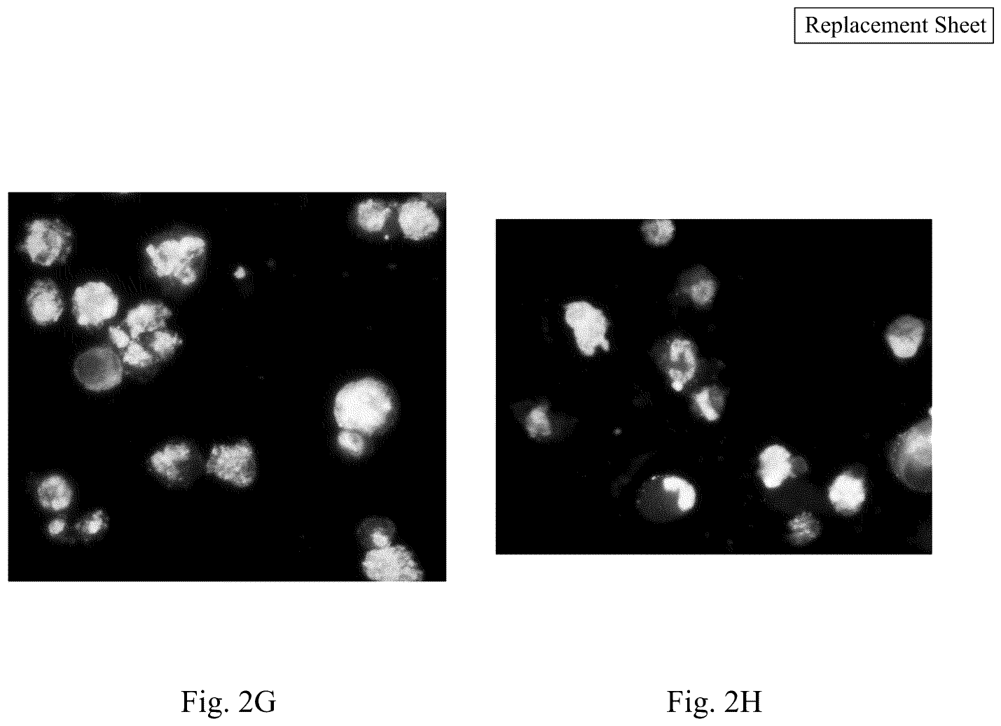

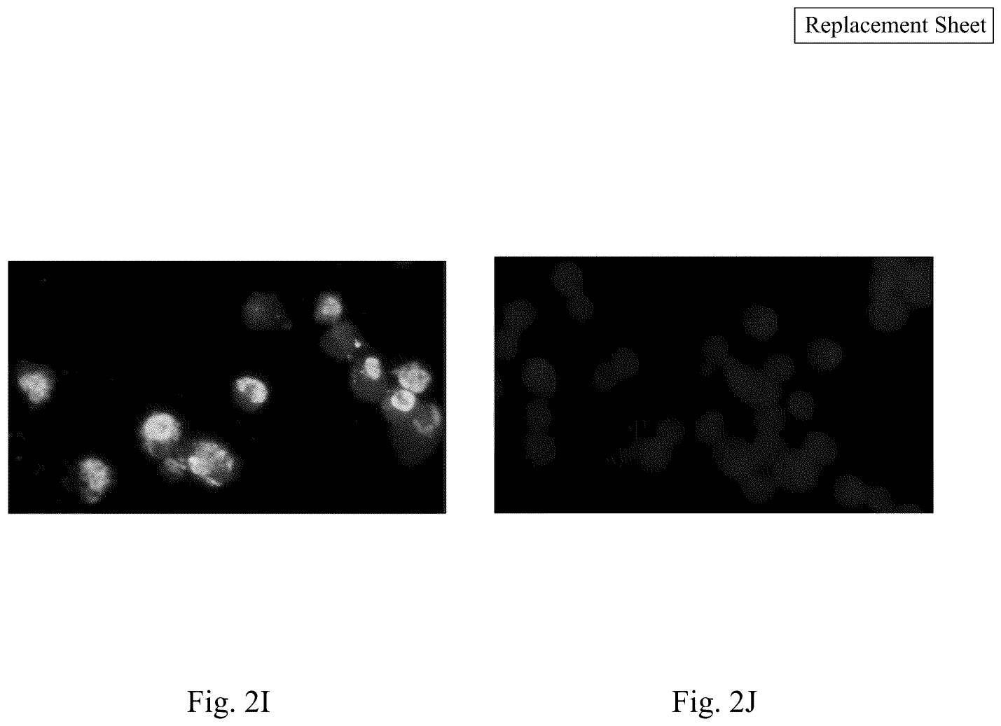

Reference is made to FIG. 2A to FIG. 2J and Table 2. FIG. 2A shows analysis result of immunofluorescence staining by the isolated monoclonal antibody of present disclosure on cells infected with dengue virus serotype 1. FIG. 2B shows analysis result of immunofluorescence staining by the isolated monoclonal antibody of present disclosure on cells infected with dengue virus serotype 2. FIG. 2C shows analysis result of immunofluorescence staining by the isolated monoclonal antibody of present disclosure on cells infected with dengue virus serotype 3. FIG. 2D shows analysis result of immunofluorescence staining by the isolated monoclonal antibody of present disclosure on cells infected with dengue virus serotype 4. FIG. 2E shows analysis result of immunofluorescence staining by the isolated monoclonal antibody of present disclosure on cells infected with Japanese encephalitis virus. FIG. 2F shows analysis result of immunofluorescence staining by the isolated monoclonal antibody of present disclosure on cells infected with Kunjin virus. FIG. 2G shows analysis result of immunofluorescence staining by the isolated monoclonal antibody of present disclosure on cells infected with West Nile virus. FIG. 2H shows analysis result of immunofluorescence staining by the isolated monoclonal antibody of present disclosure on cells infected with Zika virus. FIG. 2I shows analysis result of immunofluorescence staining by the isolated monoclonal antibody of present disclosure on cells infected with yellow fever virus. FIG. 2J shows analysis result of immunofluorescence staining by the isolated monoclonal antibody of present disclosure on mock cells. In FIG. 2A to FIG. 2J, green fluorescence signals represent signals of the isolated monoclonal antibody of present disclosure, and red fluorescence signals represent autofluorescence of the cells. Table 2 shows cross-react test results and neutralizing effect of the isolated monoclonal antibody of present disclosure, wherein “NT++” represents completely neutralizing effect, “NT+” represents partial neutralizing effect and “NT−” represents no neutralizing effect.

TABLE 2

| Flavivirus | Neutralizing effect |
|---|---|
| Dengue virus serotype 1 | 3+/NT++ |
| Dengue virus serotype 2 | 3+/NT++ |
| Dengue virus serotype 3 | 3+/NT++ |
| Dengue virus serotype 4 | 3+/NT++ |
| Japanese encephalitis virus | 3+/NT+ |
| Kunjin virus | 3+/NT+ |
| Yellow fever virus | 4+/NT− |
| West Nile virus | 3+/NT+ |
| Zika virus | 3+/NT+ |

The results in FIG. 2A to FIG. 2I show that the isolated monoclonal antibody of present disclosure can specifically bind to dengue virus serotype 1, dengue virus serotype 2, dengue virus serotype 3, dengue virus serotype 4, Japanese encephalitis virus, Kunjin virus, West Nile virus, Zika virus and yellow fever virus in flavivirus-infected cells but not in mock cells. The results in Table 2 show that the isolated monoclonal antibody of present disclosure can cross-react to all flaviviruses tested and can broadly neutralize the flaviviruses.

Therefore, the isolated monoclonal antibody of the present disclosure has excellent specific binding ability to the E protein of the flavivirus and can broadly neutralize the flavivirus. Accordingly, the isolated monoclonal antibody of the present disclosure can be used for eliciting the immune response against the antigenic epitope of the flavivirus in the subject, and can be used to detect the flavivirus. In particular, the flavivirus can be dengue virus serotype 1, dengue virus serotype 2, dengue virus serotype 3, dengue virus serotype 4, Japanese encephalitis virus (JEV), Kunjin virus, yellow fever virus, West Nile virus or Zika virus. In addition, the isolated monoclonal antibody of the present disclosure can be used as an antibody-cell conjugation for the treatment of flavivirus infection.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. An isolated monoclonal antibody, comprising:
a heavy chain variable domain (V_{H}) comprising a heavy chain complementary determining region (HCDR)1 of SEQ ID NO: 1, a HCDR2 of SEQ ID NO: 2, and HCDR3 of SEQ ID NO: 3; and
a light chain variable domain (V_{L}) comprising a light chain complementary determining region (LCDR)1 of SEQ ID NO: 4, a LCDR2 having an amino acid sequence of SAS, and a LCDR3 of SEQ ID NO: 5;
wherein the isolated monoclonal antibody specifically binds to a glycosylated envelope (E) protein of a flavivirus and neutralizes the flavivirus.

2. The isolated monoclonal antibody of claim 1, wherein the V_{H} having an amino acid sequence of SEQ ID NO: 6 and the V_{L} having an amino acid sequence of SEQ ID NO: 7.

3. The isolated monoclonal antibody of claim 1, wherein the isolated monoclonal antibody is an IgG, an IgM or an IgA.

4. The isolated monoclonal antibody of claim 1, wherein the isolated monoclonal antibody is linked to an effector moiety.

5. The isolated monoclonal antibody of claim 4, wherein the effector moiety is a toxin or a detectable label.

6. The isolated monoclonal antibody of claim 5, wherein the detectable label is a fluorescent label, an enzymatic label or a radioactive label.

7. An isolated antigen binding fragment of the isolated monoclonal antibody of claim 1.

8. The isolated antigen binding fragment of claim 7, wherein the isolated antigen binding fragment is a Fab fragment, a Fab′ fragment, a F(ab′)2 fragment, a single chain Fv protein (scFv) fragment or a disulfide stabilized Fv protein (dsFv).

9. A method of eliciting an immune response against an antigenic epitope of a flavivirus in a subject, comprising administering a therapeutically effective amount of the isolated monoclonal antibody of claim 1 to the subject, thereby eliciting the immune response.

10. The method of claim 9, wherein the flavivirus is dengue virus serotype 1, dengue virus serotype 2, dengue virus serotype 3, dengue virus serotype 4, Japanese encephalitis virus, Kunjin virus, yellow fever virus, West Nile virus or Zika virus.

11. The method of claim 9, wherein the subject is a mammal.

12. A composition, comprising the isolated monoclonal antibody of claim 1 and a pharmaceutically acceptable carrier.

13. A method of eliciting an immune response against an antigenic epitope of a flavivirus in a subject, comprising administering an immunogenically effective amount of the composition of claim 12 to the subject, thereby eliciting the immune response against dengue virus serotype 1, dengue virus serotype 2, dengue virus serotype 3, dengue virus serotype 4, Japanese encephalitis virus (JEV), Kunjin virus, yellow fever virus, West Nile virus or Zika virus.

14. A method for detecting a flavivirus, comprising:
providing a sample from a subject;
contacting the sample with the isolated monoclonal antibody of claim 1 and performing a binding reaction; and
detecting whether the sample has an antibody-virus complex.

15. The method for detecting the flavivirus of claim 14, wherein the flavivirus is dengue virus serotype 1, dengue virus serotype 2, dengue virus serotype 3, dengue virus serotype 4, Japanese encephalitis virus (JEV), Kunjin virus, yellow fever virus, West Nile virus or Zika virus.

16. A polypeptide, comprising the isolated monoclonal antibody of claim 1.

17. An antibody-cell conjugation, comprising:
a cell, wherein the cell is an immune cell or a stromal cell; and
the polypeptide of claim 16 conjugated to a surface of the cell, wherein the polypeptide specifically binds to a glycosylated envelope (E) protein of a flavivirus.

18. The antibody-cell conjugation of claim 17, wherein the immune cell is a T cell or a natural killer (NK) cell.
